(19) United States
(12) Patent Application Publication
(10) Pub. No.: US 2019/0167628 A1
(43) Pub. Date: Jun. 6, 2019

(54) METHODS FOR TREATING SARCOMA

(71) Applicant: NantOmics, LLC, Culver City, CA (US)

(72) Inventors: Sheeno THYPARAMBIL, Frederick, MD (US); Dongyao YAN, Culver City, CA (US); Shankar SELLAPPAN, Clarksville, MD (US); Fabiola CECCHI, Potomac, MD (US); Todd HEMBROUGH, Gaithersburg, MD (US); Yeoun Jin KIM, Gaithersburg, MD (US); Andrew CHAMBERS, Rockville, MD (US)

(21) Appl. No.: 16/207,878

(22) Filed: Dec. 3, 2018

Related U.S. Application Data
(60) Provisional application No. 62/593,670, filed on Dec. 1, 2017.

(51) Int. Cl. A61K 31/282; A61P 35/00; A61K 31/337; A61K 31/4745; A61K 31/7068

(52) U.S. Cl. CPC A61K 31/282; A61P 35/00; A61K 31/7068; A61K 31/4745; A61K 31/337

(57) ABSTRACT

Molecular markers of response to various chemotherapies were measured in tumor samples of 5 sarcoma subtypes to identify effective treatment regimens based on expression of chemotherapy biomarkers. The molecular profiles for rhabdomyosarcoma support the use of standard-of-care treatment, while other profiles showed that alternative agents differing from standard-of-care will be effective, including gemcitabine (osteosarcoma), taxane (leiomyosarcoma, Ewing's sarcoma), and platinum (leiomyosarcoma, liposarcoma, and osteosarcoma).

### CROSS REFERENCE TO RELATED APPLICATIONS

This application claims priority under 35 U.S.C. § 119(e) from U.S. Provisional Patent Application Ser. No. 62/593,670, filed Dec. 1, 2017, the entire contents of which are hereby incorporated by reference.

### FIELD OF THE INVENTION

Improved methods for treating sarcoma are provided.

### BACKGROUND

Sarcomas are a heterogeneous group of connective tissue cancers. Examples of sarcomas include osteosarcoma, Ewing's sarcoma, leiomyosarcoma, liposarcoma, and rhabdomyosarcoma. Anthracyclines are often the first line of treatment for the various sarcomas, but gemcitabine, taxane and other agents have shown activity. Treatment regimens for sarcoma have not to date used mechanism-based insights where treatment has been informed by molecular markers of response to tumor therapy. The methods described below describe measurements of molecular markers of response to various chemotherapies in tumor samples of 5 sarcoma subtypes. The methods were used to identify effective treatment regimens based on expression of chemotherapy biomarkers.

### SUMMARY OF THE INVENTION

What are provided are methods of treating leiomyosarcoma, which include treating a patient suffering from leiomyosarcoma with a regimen that includes an effective amount of a tubulin inhibitor and/or an effective amount of a platinum-based agent. The tubulin inhibitor may be a taxane. The platinum-based agent may be cisplatin or carboplatin. In these methods the regimen may include an effective amount of a tubulin inhibitor and an effective amount of a platinum-based agent.

Further provided are methods of treating liposarcoma, which include treating a patient suffering from liposarcoma with a regimen that includes an effective amount of a pyrimidine antagonist and/or an effective amount of a topoisomerase inhibitor. Also provided are methods of treating osteosarcoma, which include treating a patient suffering from osteosarcoma with a regimen that includes an effective amount of a pyrimidine antagonist and/or an effective amount of a topoisomerase inhibitor. The pyrimidine antagonist may be, for example, Gemcitabine. The topoisomerase inhibitor may be, for example, irinotecan. In these methods, the regimen may include an effective amount of a pyrimidine antagonist and an effective amount of a topoisomerase inhibitor. Proteomic analysis of tumor tissue from sarcoma patients was carried out to identify tumor molecular characteristics that are predictive of response or resistance to chemotherapies. The results of the analysis were surprising and were used to design new and improved methods of treatment for sarcoma patients.

### BRIEF DESCRIPTION OF THE DRAWINGS

FIG. 1 shows the proteomic landscape of various chemotherapy markers in osteosarcoma.

FIG. 2 shows the proteomic landscape of various chemotherapy markers in Ewing's sarcoma.

FIG. 3 shows the proteomic landscape of various chemotherapy markers in leiomyosarcoma.

FIG. 4 shows the proteomic landscape of various chemotherapy markers in liposarcoma.

FIG. 5 shows the proteomic landscape of various chemotherapy markers in rhabdomyosarcoma.

FIG. 6 shows proteogenomic (protein and RNA-seq) correlation of the chemotherapy marker ERCC1. For this marker there was an inverse correlation (r=−0.36).

FIG. 7 shows proteogenomic correlation of the chemotherapy marker TOPO2A. This marker has a positive correlation (r=0.86).

FIG. 8 shows proteogenomic correlation of the chemotherapy marker hENT1. This marker has a positive correlation (r=0.45

FIG. 9 shows proteogenomic correlation of the chemotherapy marker RRM1. This marker has a positive correlation (r=0.37)

FIG. 10 shows that 93% of osteosarcoma samples (n=14) expressed the response marker for gemcitabine (hENT1), while only 14% expressed RRM1 protein at a level conferring gemcitabine resistance.

### DETAILED DESCRIPTION

75 sarcoma samples were analyzed with the GPS Cancer™ (Genomic Proteomic Spectrometry) Cancer molecular diagnostic test (NantHealth, Inc., described at http://www.gpscancer.com/) The GPS test employs whole genome sequencing, whole transcriptome (RNA) sequencing, and quantitative proteomics based on mass spectrometry-based Selected Reaction Monitoring (SRM). Microdissected sarcoma tumor tissues were solubilized using the Liquid Tissue® protocol and reagents available from Expression Pathology (Rockville, Md.) and the resulting lysates injected into a mass spectrometer for quantitation of 30 biomarker proteins.

The GPS Cancer™ test includes whole genome sequencing (WGS), RNA-sequencing and targeted proteomic analysis. For the nucleic acid sequencing, tumor tissue was biopsied, and sent along with blood for preparation for omics analysis. The sample was microdissected, and DNA & RNA were extracted. For the proteomic analysis, tumor cells from formalin-fixed paraffin-embedded slides were identified by a pathologist. The cells were microdissected by non-contact laser-based dissection using DIRECTOR® slides, followed by Liquid Tissue® processing to release protein fragments into solution. Multi-protein quantitation was carried out by mass spectrometry.

Table 1 below shows the genomic features of the 75 clinical samples by sarcoma subtype:

TABLE 1
Tumor characteristics

| Tumor type | No. of sample | Genomic features |
|---|---|---|
| Osteosarcoma | 14 | MKRN2-RAF1 |
| Ewing's sarcoma | 12 | EWSR1-FLI1 |
| | | FLI1-EWSR1 |
| Leiomyosarcoma | 25 | FAM9A-BRD3 |
| | | MSH2-FEZ2 |
| Liposarcoma | 13 | MSH2-FEZ2 |
| | | NTRK3-TOP1 |
| | | TMCO1-NTRK3 |
| | | FUS-DDIT3 |
| Rhabdomyosarcoma | 11 | PAX3-FOXO1 |

Analysis of the expression of the proteins ERCC1, TUBB3, TOP2A, TOP1, hENT1/RRM1, and MGMT was carried out on the tissue. Each of these proteins has been previously suggested as being indicative of either sensitivity or resistance to treatment regimens, as summarized in Table 1 below: The molecular assessment of these potential biomarkers in sarcoma cancer subtypes was then used to guide new and improved methods and treatments compared to current standard of care regimens.

TABLE 2

| | |
|---|---|
| Marker | Agents |

Sensitivity Markers

| | |
|---|---|
| hENT1 | Gemcitabine |
| TOPO2A | Anthracyclines (Doxorubicin, Epirubicin), |
| | Etoposide |
| TOPO1 | Irinotecan, Topotecan |

Resistance Markers

| | |
|---|---|
| RRM1 | Gemcitabine |
| MGMT | Temzolomide |
| TUBB3 | Taxanes |
| ERCC1 | Platinum salts |

### Results:

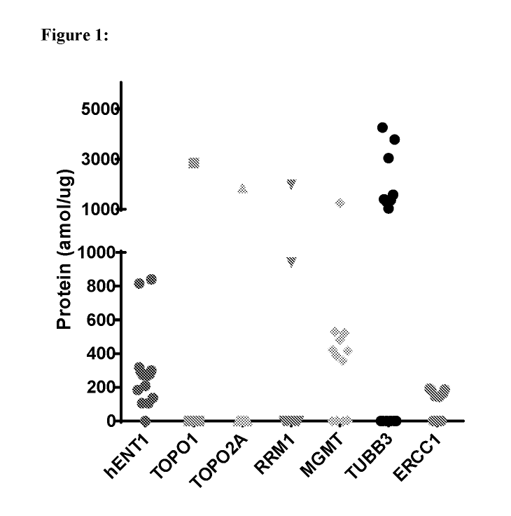

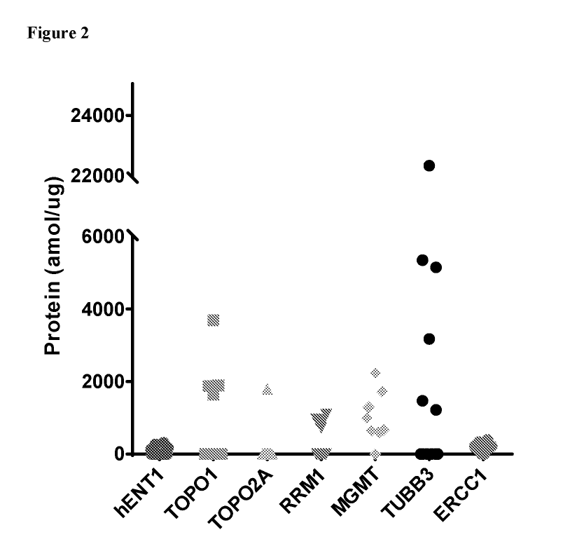

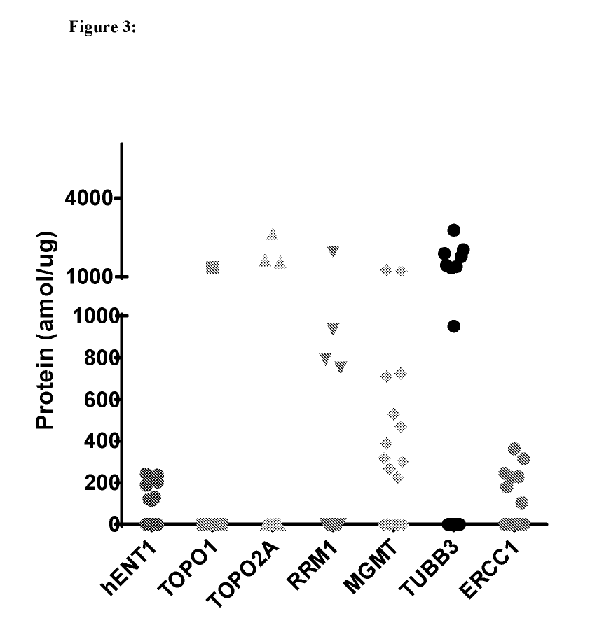

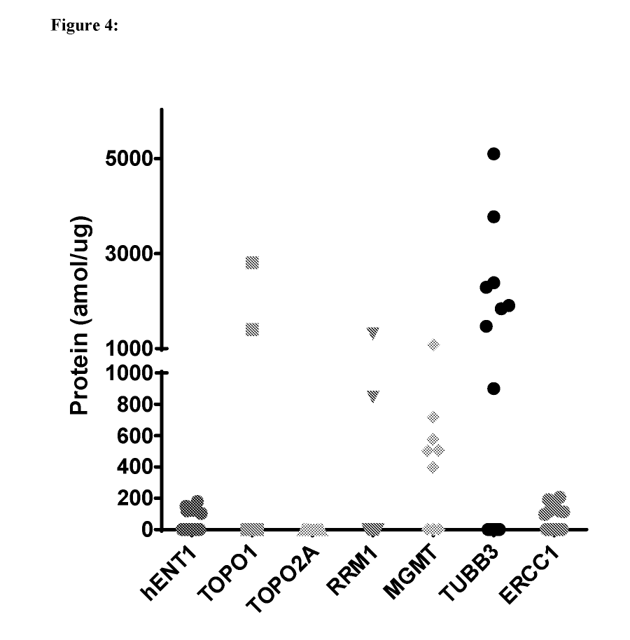

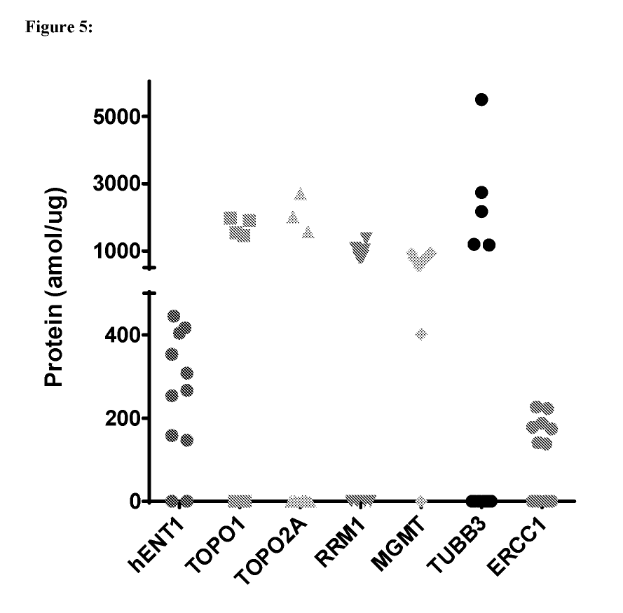

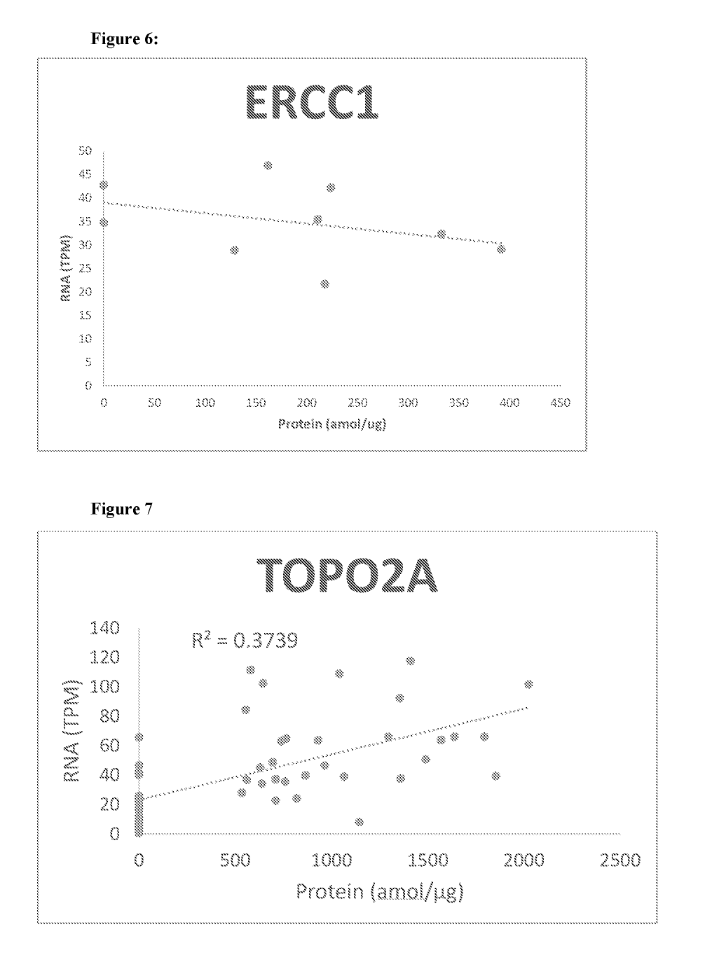

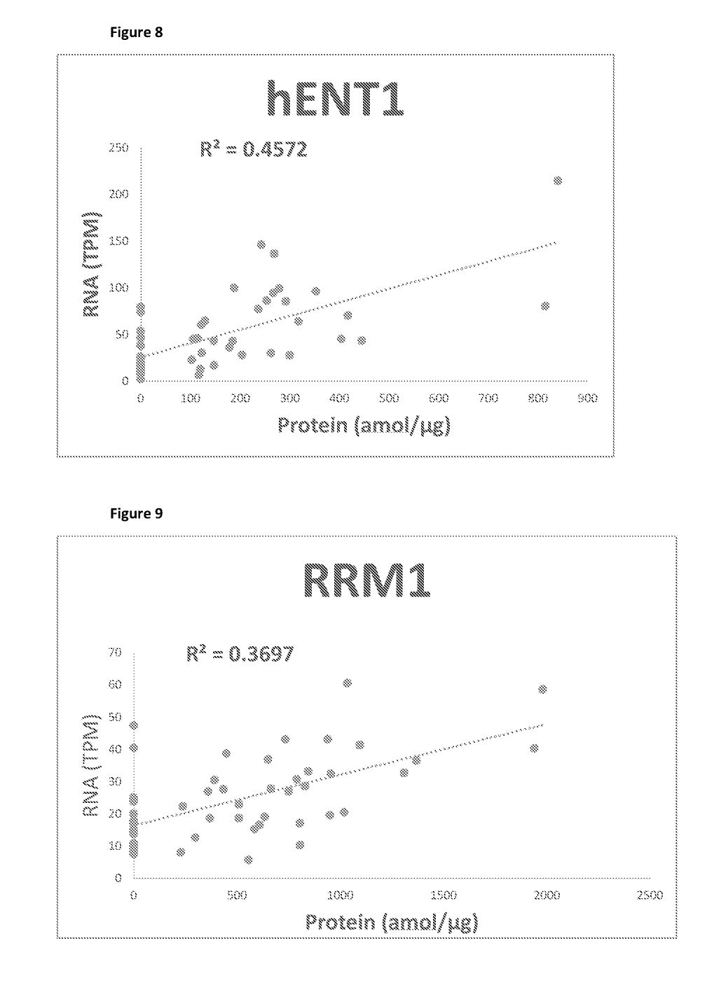

FIGS. 1-5 show the proteomic landscape of chemotherapy (sensitivity and resistance) markers for each of the sarcoma subtypes (osteosarcoma, Ewing's sarcoma, leiomyosarcoma. Liposarcoma and rhabdomyosarcoma, respectively). FIGS. 6-9 show proteogenomic correlations for the chemotherapy markers ERCC1, TOPO2A, hENT1 and RRM1 respectively. Protein and RNA-seq correlation of selected chemotherapy biomarkers are shown. Some targets were inversely correlated (e.g., ERCC1; r=−0.36), while others had positive correlation (TOPO2A; r=0.86). The data were used to analyze the likely response of patients to chemotherapy agents. Table 3 below shows the percentage of patients likely to respond to chemotherapy agents based on proteomic biomarker expression, by sarcoma subtype.

TABLE 3

| | | Ewing's | | | |
|---|---|---|---|---|---|
| | Osteosarcoma | Sarcoma | Leiomyosarcoma | Liposarcoma | Rhabdomyosarcoma |
| Topotecan/ | 7 | 33 | 4 | 15 | 36 |
| Irinotecan | | | | | |
| Anthracycline | 7 | 8 | 13 | 0 | 27 |
| Gemcitabine | 79 | 25 | 32 | 38 | 9 |
| Platinum | 54 | 17 | 72 | 46 | 36 |
| Taxane | 43 | 50 | 68 | 38 | 55 |
| Temozolomide | 33 | 12 | 42 | 33 | 12 |

### Osteosarcoma

TOPO1 protein was overexpressed in 12 of 14 (86%) osteosarcomas. hENT1 is a nucleoside transporter which enables gemcitabine to enter the cell. The effect of gemcitabine is reduced by high levels of RRM1. 93% of osteosarcoma samples (n=14) expressed the response marker for gemcitabine (hENT1), while only 14% expressed RRM1 protein at a level conferring gemcitabine resistance. Overall, ˜80% of osteosarcoma shows sensitivity to gemcitabine.

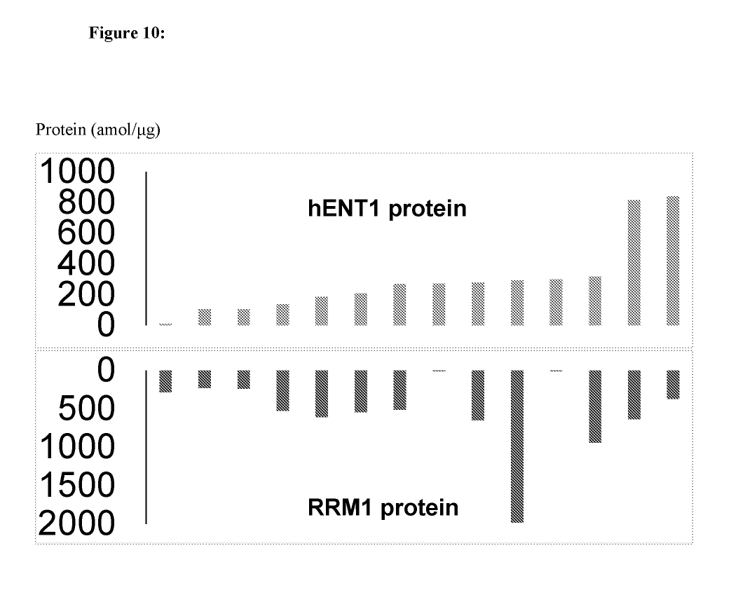

The vast majority of these samples expressed hENT1 protein and lacked expression of RRM1 protein. This proteomic signature indicates that these patients will benefit from a regimen including TOP1 inhibitors (irinotecan) and gemcitabine. The data are summarized in FIG. 10. The threshold for expression of hENT1 that conferred sensitivity to gemcitabine was >100 amol/μg, while the threshold of RRM1 that conferred resistance to gemcitabine was >700 amol/μg

The current standard of care treatment for osteosarcoma is a combination of a platinum-based agent plus and an anthracycline. The results described herein surprisingly indicate that treatment with Gemcitabine and Irinotecan is effective.

### Ewing's Sarcoma

Patients with Ewing's sarcoma (n=12) overexpressed TOPO1 (92%) and TOPO2A (58%) proteins, indicating susceptibility to Irinotecan and anthracyclines. Temozolomide has been studied as a putative treatment for Ewing's sarcoma, the results described herein suggest that this treatment will be ineffective because the majority (88%) of patients expressed MGMT, a marker for temozolomide resistance.

The current standard of care for Ewing's sarcoma is a combination of Irinotecan and an anthracycline. The results described herein indicate no change from this standard of care

### Leiomyosarcoma

18 of 25 (72%) patients with leiomyosarcoma (LMS) did not express ERCC1 protein, a DNA-repairing endonuclease, indicating susceptibility to DNA damaging agents (e.g., cisplatin, carboplatin). Similarly, 17 of 25 (68%) samples of LMS did not express TUBB3, a resistance marker for tubulin inhibitors (e.g., taxane).

The current standard of care treatment for leiomyosarcoma is a combination of Gemcitabine and an anthracycline agent. The results described herein surprisingly indicate that treatment with a taxane+a platinum-based agent is effective.

### Liposarcoma

7/13 (54%) dedifferentiated liposarcoma (DDLPS) samples expressed TOPO1 protein indicating that these patients will benefit from irinotecan-based therapy.

The current standard of care treatment for liposarcoma is Gemcitabine and an anthracycline agent. The results described herein surprisingly indicate that treatment with Gemcitabine and Irinotecan is effective.

### Rhabdomyosarcoma

In patients with rhabdomyosarcoma (n=11), TOPO1 and TOPO2A proteins were overexpressed in 91% and 82% of samples, respectively. TOPO1 and TOPO2A are response markers for irinotecan and doxorubicin. In addition, 55% of samples lacked expression of the taxane resistance marker TUBB3. Markers for gemcitabine (hENT1/RRM1) pointed to resistance, with 73% of patients overexpressing RRM1.

The current standard of care for rhabdomyosarcoma is a combination of a taxane, an anthracycline and Irinotecan and the results described herein indicate no change from this standard of care.

These data are summarized below in Table 4, which shows a comparison of standard of care and potentially effective treatments based on tumor biology.

TABLE 4

| | Osteosarcoma | Ewings | Leiomyosarcoma | Liposarcoma | Rhabdomyosarcoma |
|---|---|---|---|---|---|
| Standard | methotrexate, | vincristine, | gemcitabine, | gemcitabine, | taxane, |
| of care | anthracycline, | ifosfamide, | anthracycline, | anthracycline, | anthracycline, |
| | platinum | anthracycline, | trabectedin | trabectedin | irinotecan |
| | | Etoposide | | | |
| Potentially | gemcitabine, | taxane | taxane, | gemcitabine, | taxane, |
| effective | platinum, | | platinum, | platinum, | anthracycline, |
| | trabectedin | | trabectedin | trabectedin | irinotecan |

What is claimed is:

1. A method of treating leiomyosarcoma, comprising treating a patient suffering from leiomyosarcoma with a regimen comprising an effective amount of a tubulin inhibitor and/or an effective amount of a platinum-based agent.

2. The method according to claim 1 wherein said tubulin inhibitor is a taxane.

3. The method according to claim 1 or claim 2 wherein said platinum-based agent is cisplatin or carboplatin.

4. The method according to any preceding claim wherein said regimen comprises an effective amount of a tubulin inhibitor and an effective amount of a platinum-based agent.

5. A method of treating liposarcoma, comprising treating a patient suffering from liposarcoma with a regimen comprising an effective amount of a pyrimidine antagonist and/or an effective amount of a topoisomerase inhibitor.

6. The method according to claim 5 wherein said pyrimidine antagonist is Gemcitabine.

7. The method according to claim 5 or claim 6 wherein said topoisomerase inhibitor is irinotecan.

8. The method according to any of claims 5-7 wherein said regimen comprises an effective amount of a pyrimidine antagonist and an effective amount of a topoisomerase inhibitor.

9. A method of treating osteosarcoma, comprising treating a patient suffering from osteosarcoma with a regimen comprising an effective amount of a pyrimidine antagonist and/or an effective amount of a topoisomerase inhibitor.

10. The method according to claim 9 wherein said pyrimidine antagonist is Gemcitabine.

11. The method according to claim 9 or claim 10 wherein said topoisomerase inhibitor is irinotecan.

12. The method according to any of claims 9-11 wherein said regimen comprises an effective amount of a pyrimidine antagonist and an effective amount of a topoisomerase inhibitor.
